(19) United States
(12) Patent Application Publication
(10) Pub. No.: US 2011/0046262 A1
(43) Pub. Date: Feb. 24, 2011

(54) Pasty Insert Material for Widening of Gingival Sulcus and Use Thereof

(75) Inventors: Alexander Bublewitz, Herborn (DE); Jens-Peter Reber, Meinerzhagen (DE)

Correspondence Address: MCDONNELL BOEHNEN HULBERT & BERGHOFF LLP, 300 S. WACKER DRIVE, 32ND FLOOR, CHICAGO, IL, 60606, US

(73) Assignee: KETTENBACH GMBH & CO. KG, Eschenburg (DE)

(21) Appl. No.: 12/812,408

(22) PCT Filed: Jan. 21, 2009
(86) PCT No.: PCT/EP09/00349

§ 371 Date: Oct. 1, 2010

(30) Foreign Application Priority Data
Jan. 21, 2008 (DE) 10 2008 005 469.0

(51) Int. Cl. A61K 6/00

(52) U.S. Cl. 523/121

(57) ABSTRACT

Described are new pasty masses that are suitable as insert material for widening the gingival sulcus. These retraction materials contain a paste-forming agent, superabsorber particles and at least one astringent.

Surprisingly, it was shown that upon contact with fluid of such a retraction material paste formulation, for one, a volume expansion is occurs due to absorbed fluid and for another, a part of the superabsorber is diffused out of the paste and/or dissolved out and that this diffused and/or dissolved part in turn leads to the immediate thickening, bonding and/or coagulation of surrounding fluids such as saliva, blood and wound secretions. The effect is hemostasis, as well as widening of the sulcus.

New pasty masses are described that are suitable as insert material for widening the gingival sulcus (hereinafter also “retraction material).

Retraction materials are used in dentistry for opening the sulcus and for hemostasis in the sulcus prior to taking the actual impression of prepared teeth.

In dental care, for example, by means of a crown, the tooth must be ground with a rotating instrument. For that, a preparation boundary must be ground into the tooth. Its purpose is to define a precise margin fit for the crown that is to be inserted. For aesthetic reasons, an attempt is made to place the margin fit as much as possible in the section of, or underneath the gingival sulcus margin. During the preparation with the rotating instrument, the gingival is often injured or it is parodontally damaged and bleeds for these reasons. In addition, the gingival sulcus is additionally moistened by the flow of saliva.

The big challenge for the dentist now is, to make an exact impression of the ground tooth with the preparation boundary under these unfavorable conditions. In order to ensure that the impression provides a good result, during the preparation, for one, the sulcus is widened with a so-called retraction material, and for another, the hemostasis is stopped by astringent additives.

In prior art, many retraction materials have already been described. Thus, different retraction threads are known (DE-A-10 2004 032 099, EP-A-317,690), or retraction pastes are used, (WO-A-96/27342, DE 690 06 030 T2), or retraction silicon foam materials are used (US-A-2005/0202367, US-A-2005/0069838).

From DE 69006030 T2 as well as U.S. Pat. No. 5,362,495, retraction materials are known that are provided with astringent substances such as aluminum potassium alum.

DE-A-37 37 552 describes a fluid, ductile combination of substances that leads to a swelling pressure in the sulcus by an increase in volume due to a chemical reaction, physical swelling effects or as a result of both properties. As impression base materials, alginates, hydrocolloids as well as silicones are mentioned as being preferred.

Even in EP-A-317,690, the physical swelling effect of a thread material, here threads consisting of superabsorber are utilized in order to produce a swelling pressure in the sulcus and thus a widening of the sulcus.

DE 37 36 155 A1 describes a ductile, fluid dental impression material that is mixed with pharmacologically active ingredients for retracting the gingival margin, for drying up and for hemostasis. As a preferred material, a mixture of fine alginate powder with fluid pharmacologically active ingredients and water is mixed into a liquid-malleable impression mass.

DE 101 03 446 A1 describes multi-component systems for making impressions of hardenable materials resulting from mixtures. These are combinations of crosslinking silicones due to addition and due to condensation. Superabsorbers such as alkali salts of poly(meth)acrylic acid can be added as drying agents.

DE 696 34 803 T2 reveals a hemostatic composition for use in dental procedures, specifically for use in the gingival region of the tooth. This composition essentially consists of a selected hemostatic remedy, an inorganic filler substance and/or a polyol with high molecular weight, as well as a watery base, and which has a hemostatic and retraction-facilitating effect when the impression is taken.

US 2008/0220050 A1 describes pasty retraction materials that contain water, clay, micronized filler substance based on glass and astringents.

WO 2008/012740 A2 describes a hardenable retraction material in the form of a paste based on alginate, two or three-valent ions, water, retarder and filling agent.

When using previously known retraction materials, frequently, problems occur. Thus, retraction threads, for example, retraction threads saturated with astringents are very difficult to place in the sulcus, at great expense of time. Although these retraction threads lead to widening and hemostasis, they must be removed again prior to making the impression. As a result of this, the sulcus starts to bleed again in most cases so that here, bad impression conditions exist most of the time.

In the case of the silicone foams used, a hemostasis is achieved only by means of the arising compression pressure. The widening of the sulcus is to take place as a result of the pressure of the expanding foam. Even this is not solved satisfactory, as the foam seeks the path of least resistance and pushes upward out of the gingival pocket and expands there. These silicone retraction materials crosslink after being inserted into the sulcus and can subsequently be removed out of the sulcus in one piece. However, there is the danger that residuals of the material remain in the sulcus, as the material flows out very fine and forms fine flags that can tear easily.

When using retraction pastes according to DE 69006030 T2, problems can occur, as the necessary rinsing out of the paste prior to taking an impression is not satisfactorily solved. Prior to an impression, the retraction paste must be rinsed out of the sulcus with the air/water blower. Because of the compact consistency of the retraction paste, this is frequently done only inadequately and can therefore, because of the required intensive air/water blower conditions, frequently lead to subsequent bleeding. Moreover, the retraction pastes described in DE 69006030 T2 contain algae meal or clay, which only have an insufficient ability to swell and therefore cannot make a contribution to the widening of the gingival sulcus. An additional disadvantage of these retraction materials is the quick drying of the materials, for example, the quick drying of the material after the package is opened. Because of the relatively high water content, special packaging systems are also required.

Thus, there continues to be a need for an improvement of previously known retraction materials.

Pastes based on superabsorbers have already been described. From WO 2005/037,894 A1, a paste of this type is known. It contains up to 5% by weight particles of superabsorbent polymer and at least 95% by weight of a watery polymer solution. As superabsorbent polymers, crosslinked polymers that are derived from ethylenically unsaturated acids, for example, acrylic acid or methacrylic acid with a particle size of 150 to 850 μm are described. The watery polymer solutions contain up to 5% by weight solid substances, which are preferably water-soluble precursors of superabsorbent polymers, for example, polyacrylic acid. The pastes are used for coating substrates or as drilling aids. Applications in dentistry are not revealed.

Based on this prior art, the problem consists of providing a paste-like retraction material that is non-continuous, not very sticky, easy to apply as homogeneous strand and that can be dissolved away again easily, for example, by using an air/water blower, which upon use widens the sulcus, as well as thickens and binds fluids such as blood and saliva that are in the area surrounding the retraction material.

Surprisingly, it was found that the retraction material in accordance with the invention thickens and binds the surrounding fluid in addition to the actual swelling effect.

The present invention concerns a pasty retraction material for widening the gingival sulcus containing a) paste-forming agents, b) superabsorber particles, and c) at least one astringent additive.

Surprisingly, it has been shown that such a retraction material paste formulation upon contact with fluid causes, for one, a volume expansion due to absorbed fluid and for another, a part of the superabsorber diffuses out of the paste and/or is dissolved out and that this part that is diffused out or dissolved out in turn leads to, that the surrounding fluid such as saliva, blood and wound secretion is immediately thickened, bound and/or coagulated.

In this way, the hemostasis as well as the widening of the sulcus is supported, i.e. blood and saliva are thus bonded at the site and do not flow away uncontrolled in all directions.

As an additional advantage of the retraction material in accordance with the invention that contains superabsorber particles b) and perhaps additional fluid absorbers e), this retraction material can be adjusted to a thinner viscosity than that which is revealed in prior art.

This has the following advantages with respect to prior art: For one, as a result of the thinner viscosity, an easier manual application using known delivery equipment is achieved, so that shaking and thus the risk of injury can be avoided, and for another, the diameter of the needle of the syringe of the application system can be comparably smaller and thus an application of the paste to the sulcus that is gentle to the patient is made possible.

But on the other hand, due to the fluid absorption and the swelling and expansion of the superabsorber b) that is connected with it and the perhaps present fluid absorber e), a sufficiently high swelling pressure and a sufficient sulcus retraction or enlargement is achieved.

The pasty retraction material in accordance with the invention preferably has a measured viscosity that is larger than 13,000 Pa s at 20° C.

In an additional embodiment of the paste in accordance with the invention, biocompatible, volatile solvents/substances can be used which, as a result of the heat loss due to evaporation, compensate for one the heat that is released by the absorption of ingredient substances b) and e) and which beyond that create a cooling and thus vasoconstrictive effect.

After contact with the fluid from the sulcus tissue, the swollen superabsorber particles can easily be removed from the surface of the material with the air/water blower or water-soluble astringents can easily be dissolved or coarse filler substances can be removed easily, whereby craters are formed that facilitate an additional attack of the air/water blower and in this way lead to an easy rinsing out of the retraction material from the sulcus.

In this way, the paste that is sitting deep in the sulcus is also rinsed out again easily, without requiring work that is too lengthy or with water pressure that is too high, which would otherwise again lead to a mechanical irritation and bleeding of the sulcus, which is to be avoided at all cost.

Paste-forming materials can be used as ingredient a), which preferably have a hydrophilic consistency and which, in combination with ingredients b) and c) as well as perhaps additional additives, make the formulation of a paste possible. Thereby, these are substances that are liquid in the temperature range of processing and application, i.e. are usually liquid in a range between 20 and 40° C., and which have the compatibility that is required for the application.

Preferably, ingredient a) is selected in such a way that it leads to a pliable, not crumbly consistency of the retraction material, and can preferably be eluted with water.

Examples of paste-forming agents are polysiloxanes, polyalkylene oxides, multi-valent, especially bivalent or trivalent aliphatic alcohols, esters or half-esters of the identified bi, tri or multi-valent alcohols with saturated and/or unsaturated carboxylic acids, liquid hydrocarbons or water.

Preferably, the paste-forming agents a) are polysiloxanes, especially preferred polydialkyl siloxane, polyalkylene glycols, in particular, polyethylene glycols and/or polypropylene glycols and/or polybutylene glycols; as well as bi-valent aliphatic alcohols such as 1,2-propanediol, 1,3-propanediol, 1,2-ethanediol, butanediol or hexanediol; or tri or higher-valent aliphatic alcohols, in particular triols, such as glycerin and/or trimethylol propane or tetrols, pentols or hexols, such as, for example, sorbitol, as well as alkoxylated derivatives of the previously mentioned alcohols, in particular the ethoxylated types; as well as esters or half-esters of the mentioned bi, tri or multi-valent alcohols with saturated and/or unsaturated carboxylic acids, for example, glycerin esters such as acetyltributyl citrate, such as mineral oils; and in particular water or combinations of water with polyalkylene glycols and/or with bi or multi-valent alcohols. Preferably, anhydrous pastes can also be formulated with the above mentioned paste-forming agents.

The weight proportion of ingredient a) relative to the total mass of the retraction material is customarily between 1 to 90%, preferably 5 to 70%, and especially preferred, 10 to 50%.

As ingredient b), any superabsorber particles can be used as volume-enhancing additives, to the extent these are available in particulate form. These can also be inorganic and in particular organic materials. Superabsorbers are characterized thereby, that they absorb a multiple of their own weight when in contact with water and thereby swell. For purposes of this description, superabsorber refers to a material that can absorb at least twice, preferably five times its weight and especially preferred ten times its weight of water at room temperature.

Preferably, ingredient b) is selected in such a way that it consists of micronized and/or nanoparticulate superabsorber particles and that these have a moisture-absorbing and/or swelling and/or environment or surface-fluid-binding and/or clotting and/or coagulating effect.

Examples of superabsorbent materials can be found in EP-A-317,690. In the composition in accordance with the invention, ingredient b) is present in particulate form. Typically, ingredient b) is a powder with an average particle size in the range of 50 nm to 300 μm, preferably 100 nm to 200 μm, especially preferred smaller than 150 μm, and very especially preferred 1 μm bis 100 μm.

Preferred ingredient substances b) are polymerizates that are derived from the radical polymerization of one or more ethylenically unsaturated acids and perhaps additional comonomers that can be radically polymerized with it such as monomers with ester, amide or anhydride groups. The share of the ethylenically unsaturated acids in the polymer typically amounts to at least 55% by weight, preferably 55 to 99.9% by weight.

Examples of the ethylenically unsaturated acids are ethylenically unsaturated mono or dicarboxylic acids such as acrylic acid, methacrylic acid, itaconic acid, maleic acid or fumaric acid, or ethylenically unsaturated sulfonic acids, such as vinylsufonic acid or 2-acrylamido-2-methylpropane sulfonic acid.

As comonomers, preferably the alkyl esters or anhydrides of these carboxylic acids or sulfonic acids are used.

Preferably, at least 50%, in particular at least 75% of the acid groups in the polymer are carboxylic acid groups. These are usually at least 25 mol %, preferably between 25 and 80 mol % neutralized, and are present, for example, as alkali, alkaline earth or ammonium salts.

Especially preferred, the preferred polymerizates of ingredient b) are crosslinked. This is achieved as a result of the copolymerization in the presence of small quantities of crosslinking monomers (”crosslinkers”). The proportion of crosslinkers usually is 0.001 to 5% by weight relative to the polymer. Typical crosslinkers are ethylenically unsaturated monomers that have at least two ethylenically unsaturated groups in the molecule or that have at least one ethylenically unsaturated group and at least one additional one for crosslinking, an enabling group in the molecule, for example, as a result of complex formation.

Examples of crosslinkers are at least double unsaturated amides such as methylene-bis-acryl amide or methylene-bis-acryl-methacryl amide or such as ethylene-bis-acryl amide; or at least double unsaturated esters of polyols with ethylenically unsaturated acids such as di(meth)acrylate or tri(meth)acrylate of butanediol, ethylene glycol or of polyglycols such as polyethylene glycol, or of trimethylol propane; or at least—at least double functionalized allyl compounds such as allyl(meth)acrylate, triallyl cyanurate, triallyl diisocyanurate, maleic acid diallylester or triallyl amine.

Compounds of this type are known from WO 2005/037,894 A1.

Particularly preferred ingredients b) are salts of polyacrylic acid and/or poly(meth)acrylic acid and in turn especially preferred the potassium, ammonium and/or sodium salts or partially neutralized poly(meth)acrylic acids. Preferably, these are present in turn in micronized or nanoparticulate form and preferably have an adsorption of distilled water of 20 to 1,000 g/g, as well as a swelling size of 1 to 10 mm (Source: Technical Data Sheet “Swelling Powder” from Stockhausen GmbH & Co. KG).

In place of this or in combination with these preferred substances, preferably also other swellable, very hydrophilic and powder-like substances can be used as ingredient b).

It is surprising that as a result of the use of superabsorber particles b) and perhaps additional liquid absorbers, in the pasty, pliable retraction material according to the invention, a pliable, easy to apply consistency of the material can be achieved with the advantage, that a thinner viscosity can be selected than that which is revealed in prior art. In contrast to prior art, this has the following advantages: For one, as a result of the thinner viscosity, an easier manual application using known delivery equipment is achieved, so that shaking and thus the risk of injury can be avoided, and for another, the diameter of the needle of the syringe of the application system can be comparably smaller, for example, in the range of 0.2 to 2 mm, in particular from 0.2 to 1.5 mm, and very especially preferred 0.2 to 1 mm, and thus an application of the paste to the sulcus is made possible that is gentle for the patient.

But on the other hand, as a result of the fluid absorption and the swelling and expansion of the superabsorber b) that is connected with it and perhaps additional fluid absorbers, a sufficiently high swelling pressure and a sufficient sulcus retraction or enlargement is achieved.

The weight proportion of ingredient b) relative to the total mass of the retraction material is customarily between 0.1 to 90%, preferably 1 to 70% and especially preferred 2 to 50%.

x

Astringent additives are used as ingredient c). Thereby, these can be hemostatics that act vasoconstrictive and/or hemostatically upon the affected sulcus tissue, such as water-soluble aluminum salts, for example, potassium alum, aluminum chloride, aluminum chlorate, dialuminum pentahydroxy chloride, aluminum hydrochloride, aluminum sulfate, aluminum oxalate, aluminum acetate, aluminum lactate, aluminum trifluormethane sulfonate, aluminum nitrate, aluminum ammonium sulfate, as well as manganese salts or bismuth salts such as potassium permanganate, bismuth nitrate or bismuth salicylate, or other basic, acidic or amphoteric water-soluble aluminum salts; or iron or zinc salts such as, for example, zinc chloride, zinc oxide, zinc salicylate, zinc stearate, zinc sulfate, zinc phosphate, zinc hexafluoro silicate, zinc phenol sulfonate, iron chloride, iron sulfate; or alkali halogenides, such as sodium chloride.

Additional examples of astringent additives c) are aluminum acetate, epinephrine, adrenalin, noradrenalin, ephetomin, privin, otriven, ethacridin, negatol, oxichinolin, tannic acids, tannin or other vasoconstrictors; further hydrogen peroxide, carbamide peroxide, peroxo salts or peroxo compounds or polyaziridine hydrochloride.

Potassium alum is particularly preferred as astringent additive c) as it, in addition to the hemostatic effect due to the aluminum ions, has an additional desensitizing effect because of the potassium ions. Further, potassium alum is highly biocompatible, which is known from many applications as deodorant or as hemostatic remedy.

Astringent additives c) and in particular potassium alum and/or aluminum chloride can be mixed into the paste in accordance with the invention as solution in nanoparticulate form or in micronized, finely ground crystallites or in crystal form.

Further, as astringent additives c), calcium salts can be used that have a water-solubility of at least 0.5 g/liter at room temperature. Particularly preferred are calcium sulfate, calcium sulfate dihydrate, calcium sulfate hemihydrate, calcium hydroxide, calcium hydrogen phosphate, calcium lactate, calcium gluconate and calcium acetate, as well as calcium salts of amino acids, aldonic acids and uronic acids or calcium alginate.

The weight proportion of ingredient c) relative to the total mass of the retraction material is customarily between 0.1 to 60%, preferably 0.1 to 50% and particularly preferred 2 to 50%.

The retraction material in accordance with the invention can, in addition to the ingredients a), b) and c) described above, also contain additional ingredients which give this material a desired and advantageous property.

The weight proportion of these additional ingredients, relative to the total mass of the retraction material is usually 0.1 to 90%, preferably 1 to 60% and particularly preferred 2 to 50%.

Any type of organic and/or inorganic filler substances can be used as ingredient d), to the extent these have a particle size of at least 1 μm and preferably can be explemented by means of a dental air/water blower.

Preferably, the filler particles of ingredient d) have a particle size range between 1 μm and 200 μm, preferably between 1 μm and 100 μm, and especially preferred of 5 μm and 75 μm. As a consequence of this comparably coarse particle size, after the application, easy washing out or rinsing out is ensured with dental air/water blowers.

It was surprisingly found that the air/water jet stream leads to a tearing out of the above mentioned coarse filler particles. In this way, the paste that is sitting deep in the sulcus is also again rinsed out easily, without requiring work that takes too long or with water pressure that is too high, which otherwise would again lead to a mechanical irritation and bleeding of the sulcus, which is to be avoided at all costs.

Examples of a preferred material for ingredient d) are aluminum acetate, silicates, sheet silicates, thereof in particular kaolinite, smectite, illite, chlorite, talcum, vermiculite, sauconite, saponite, nautronite, montmorillonite and mica, as well as cristobalite, quartz, wollastonite, aluminum oxide, aluminum hydroxide, dental glasses, zinc oxide, titanium oxide, rare earth oxides and rare earth fluorides, barium sulfate, zirconium oxide, polymer powder, in particular polyethylene powder, polypropylene powder, polymethyl methacrylate powder, polyurethane powder and/or solid silicone resin; preferred are X-ray opaque and fluoride-releasing substances. With the help of X-ray opaque filler substances, residuals of the retraction material potentially remaining in the sulcus can be seen using an X-ray analysis. An example of fluoride-releasing filler substances are fluor aluminum silicate glasses. Examples of X-ray opaque filler substances are the oxides of the metals Zn, W, Ag, Yb, Y, Gd, Zr, Sr, Ba, Ta, Nb, Mo, Sn, Y, La, Ce, Pr, Nd, Sm, Eu, Gd, Tb, Dy, Ho, Er Tm, Yb, Lu, Zr, in particular zirconium dioxide, strontium oxide, bismuth oxide, lanthanum oxide, zinc oxide, yttrium oxide, ytterbium oxide, tungsten oxide, niobium oxide, tantalum oxide, molybdenum oxide, as well as sulfates, in particular barium sulfate, strontium sulfate, bismuth sulfate, carbonate such as, for example, bismuth oxycarbonate and strontium carbonate, the silicates such as, for example, zirconium silicate as well as carbides and phosphates. Additional examples are ytterbium fluoride, bismuth oxychloride, calcium tungstate, barium and strontium glasses, as well as finely distributed metals such as zinc, tungsten, silver, ytterbium, yttrium, gadolinium, zirconium, strontium, tantalum, niobium, molybdenum, lanthanum or their alloys.

The weight proportion of ingredient d), relative to the total mass of the retraction material usually is 0 to 90%, preferably 5 to 70%, and especially preferred 10 to 50%.

Especially preferred filler substances are those that also have astringent properties. These can, for example, be water-soluble astringents. Here, water solubility means solubility in water of at least 130 g/l at 20° C. Examples of such ingredients are potassium alum or aluminum chloride.

Fluid absorbers for the initial absorption of the body fluids saliva, blood and wound secretions can be used as ingredient e), such as zeolites, for example, potassium, calcium, sodium, aluminum silicate, in particular zeolites with a water absorption of up to 30%, preferable with a pore size between 3 and 13 Å, very specially preferred between 3 and 5 Å; additionally silica gels, for example, blue gel, anhydrous alkaline earth sulfate and metal sulfate, for example, calcium sulfate, aluminum sulfate, magnesium sulfate such as sheet silicates. These above mentioned compounds distinguish themselves thereby, that they do not have any noticeable volume expansion upon absorbing water and take up the water into the crystal structure or cavities. Additional examples of ingredients e) are those compounds that have a certain water absorption and thus also a certain swellability, for example, a water absorption of 10 to 50% by weight. Examples of such ingredients e) are maltodextrines, alginates, collagens, locust bean gum, chitosanes, agar-agar, carrageenans, guar gum, acacia gum, succinoglycane gum, guar, tragacanth, karaya gum, xanthan, pectins, cellulose and their ionogenic and non-ionogenic derivatives such as, for example, carboxymethyl cellulose, hydroxyethyl cellulose or methylhydroxypropyl cellulose, hydrophobically modified celluloses, starch, starch ether and modified starch, as well as higher molecular polyethylene glycoles.

The weight proportion of ingredients e), relative to the total mass of the retraction material is customarily between 0 to 80% by weight, preferably 0.1 to 70% by weight and especially preferred 1 to 60% by weight.

Desensitizers can be used as ingredient f) that have desensitizing metal ions and/or due to closing of the opened dentinal tubules by means of nanoparticles and/or due to the mineralization of the hard dental substance by means of fluoride ions, influence the nerve impulse. Examples for this are potassium and strontium salts, for example, potassium chloride, potassium sulfate, potassium carbonate, potassium citrate, potassium acetate, potassium nitrate, strontium chloride, strontium nitrate, strontium citrate, strontium acetate, strontium lactate and eugenol.

Additional examples of desensitizers are fluorides. These are used in particular for combating and for the prophylaxis of caries. These fluorides are customarily used in a quantity of 0.1-0.5% by weight fluorine, relative to the paste. Particularly preferred is the use of sodium fluoride, potassium fluoride, tin fluoride or monofluoro phosphates, such as disodium monofluoro phosphate (Na₂PO₃F), dipotassium monofluoro phosphate or the fluoride of an organic amino compound, for example, Olaflur® (manufacturer: Gaba).

Additional desensitizing substances f) for the retraction material in accordance with the invention are diffusible nanoparticles in isolated, aggregated and agglomerated form, which can be moistened with water, moisture, saliva and/or blood for closing the opened dentinal tubules. The diameter of the dentinal tubules is approximately 0.8 μm (compare van Meerbeck et. al., Operative Dentistry, Suppl. 5, 1992, 111-124). Examples of this are difficult to dissolve or insoluble alkali, alkaline earth and metal phosphates, their polyphosphates, tripolyphosphates, hexametaphosphates, pyrophosphates, hydroxyl phosphates, fluoride phosphates, carbonate phosphates, hydroxyl fluoride phosphates, oxides, silicates, phospho silicates, fluorides, titanates, such as, for example, micronized and preferably nanoparticulate calcium hydroxy phosphate (Ca₅[OH(PO₄)₃]), calcium fluoro phosphate (Ca₅[F(PO₄)₃]), fluoride-doped hydroxyl apatite represented by the general formula Ca₅(PO₄)₃(OHₓF_{y}) where x and y=1 and calcium fluoride (CaF₂). Very especially preferred are hydroxyl apatite and/or fluoro apatite, calcium phosphate, amorphous calcium phosphate, calcium sodium phospho silicate, alkali and alkaline earth silicates, metal oxides such as SiO₂, ZrO₂, ZnO, TiO₂, as well as aluminum cerium oxide, silica gel functionalized with ammonium chloride, aluminum trifluoride, aluminum metaphosphate, aluminum oxide, aluminum silicate, aluminum titanate.

Amorphous calcium and/or strontium phosphates can also be formed in situ during the application in the mouth. To do so, water-soluble alkali phosphates, tetraalkali pyrophosphates, dialkali hydrogen phosphates and alkali dihydrogen phosphates are added to the retraction material, which are released with the calcium from saliva, calcium ions from the hydroxyl apatite of the teeth or with calcium and/or strontium ions from added encapsulated calcium ion-containing solvents or, in the case of two-component systems, from the second component due to mixing, lead to the precipitation of the corresponding nanoparticulate insoluble phosphates. For this, the phosphate ions can also be present in encapsulated form. Examples of suitable soluble phosphates are tetrasodium and tetrapotassium pyrophosphate, disodium and dipotassium hydrogen phosphate, as well as sodium and potassium dihydrogen phosphate. Preferred are especially tetrapotassium and tetrasodium pyrophosphate, disodium and dipotassium hydrogen phosphate, sodium and potassium hydrogen phosphate, amorphous calcium phosphate (ACP), amorphous calcium phosphate fluoride (ACPF), amorphous calcium carbonate phosphate (ACCP) amorphous calcium carbonate phosphate fluoride (ACCPF), amorphous strontium phosphate (ASP), amorphous strontium calcium phosphate (ASCP), amorphous strontium calcium carbonate phosphate (ASCCP) and amorphous strontium calcium carbonate phosphate fluoride (ASCCPF).

The nanoparticles that act desensitizing can, in a further embodiment of the invention, also be created in situ. To do so, for example, silane and/or metal alkoxides can be added to the retraction material that abreact into alcohol and nanoparticulate metal oxide upon the addition of water from saliva to the retraction material. Examples for this are titanium tetraalcoholates such as titanium tetraisopropylates or titanium tetra-n-butylate as well as zirconium tetraalcoholates such as zirconium tetrapropylate or zirconium tetrabutylate, which quickly lead to nanoparticulate titanium oxide or zirconium dioxide upon the addition of water. By using organically functionalized silanes, titanates and/or zirconates, functional groups can be built into the structure of the nanoparticles.

The weight proportion of ingredient f), relative to the total mass of the retraction material is customarily 0 to 80%, preferably 0.01 to 60% and particularly preferred 0.01 to 40%.

Additional examples of possible additives are paste thickeners g) for selecting the viscosity, consistency and rheology such as, for example, the flow limit, thixotropy, structure viscosity. Examples of ingredient g) are pentaerythrite, cellulose derivates, polyvinyl pyrrolidone, polyvinyl alcohols, poly(meth)acrylate, xanthan, polyacrylic acid (Carbopol®) and their salts, esters and derivatives, mineral oil waxes, polyolefin waxes, polyisobutene waxes, polyether waxes, triglycerides and/or diglycerides.

Additional examples for possible paste thickeners g) are thickening organic and/or inorganic filler substances. Within the scope of this description, these are to be understood as highly dispersed, active fillers with a BET surface of at least 50 m²/g.

Preferably, the thickening filler is present as nanoparticulate fiber or flake-like filler substance, for example, as mineral fibrous filler or as synthetic, fibrous filler.

Especially suited are those paste thickeners g) that have an individual particle size in the range of nanometers, preferably in the range of 7-40 nm, which can be present as aggregates (composites of crystallites that are grown together and positioned two-dimensionally adjacent to each other) preferably in the range of 100-800 nm, and/or as agglomerates (composite that has not grown together, for example, crystallites and/or aggregates that are positioned adjacent to each other, for example at the corners or edges) preferably in the range of 10-200 μm. Preferred are thickening fillers selected from the group consisting of aluminum hydroxide, zinc oxide, titanium dioxides, zirconium oxide, silicon dioxide, precipitated silica, pyrogenic silica, sheet silicate (Bentonite®, Optigel®), and combinations of such, whereby surface-coated thickening fillers such as, for example, silanized silica can be used.

Of course, the previously identified compounds can be used individually or in any combination with each other and that in hydrophilic as well as in hydrophobic form. Further, the at least one thickening filler can preferably be present as nanoparticulate fiber or flaky filler, for example, as mineral fibrous filler or as a synthetic fibrous filler.

Thereby, the nanoparticles are preferably made of water glass by means of a sol-gel method and are mono-dispersed (not aggregated or agglomerated) spherical silanized SiO₂ nanoparticles (or TiO₂ and/or ZrO₂ nanoparticles) with a very narrow particle size distribution between 5 and 200 nm, preferably 5 to 100 nm and especially preferred 5 to 50 nm.

In a particularly preferred embodiment of the retraction material in accordance with the invention, the adjustment of the viscosity, consistency and rheology occurs without the addition of paste thickeners, but as the result of a suitable composition of ingredient a), b) and c).

The weight proportion of ingredient g) relative to the total mass of the retraction material is customarily between 0 to 90%, preferably 0 to 70% and especially preferred 0.1 to 50%.

As ingredient h), solvents can be used that can contribute to the viscosity and consistency adjustment and/or to achieving analgesic cooling properties as the result of evaporative heat loss and/or to compensate heat that is released during the absorption processes. Examples of this are alcohols, e.g. ethanol or isopropanol; or ketones, e.g. acetone or ethyl methyl ketone; or ether, such as, for example diethyl ether, ethyl methyl ether or divinyl ether; or volatile hydrocarbons such as, for example, pentane, hexane; or also other solvents such as propylene carbonate, as well as water.

In a particularly preferred embodiment of the paste in accordance with the invention, biocompatible, volatile solvents or substances are used. For one, these compensate, as a result of the heat lost by evaporation during or after the application of the paste, the heat that is released during the absorption by ingredients b) and e), and beyond that, they have a cooling and thus vasoconstrictive effect.

The weight proportion of ingredient h) relative to the total quantity of the retraction material is customarily between 0 to 40% by weight, preferably 0.1 to 30% by weight and especially preferred 1 to 20% by weight.

Additional examples of possible additives are disinfecting substances i) for avoiding subsequent infections of the sulcus tissue. Examples for this are phenols, e.g. phenol, recorcinols, and/or bisphenols; or such as salicyl anilides and amides as well as their halogenated derivatives, halogenated carbanilides and p-hydroxybenzoic acid esters.

Among the disinfecting substances i), those antimicrobial ingredients are particularly suitable that curb the growth of plaque bacteria. Among these are, for example, halogenated diphenyl ethers, such as 2,4-dichloro-2′-hydroxydiphenyl ether, 4,4-dichloro-2′-hydroxydiphenyl ether, 2,4,4′-tribromo-2-hydroxydiphenyl ether, 2,4,4′-trichloro-2′-hydroxydiphenyl ether(triclosan) as antimicrobial active ingredients. In addition to bromochlorophene, bisbiguanides such as chlorohexidine, chlorohexidine gluconate, alexidine, such as p-hydroxybenzoic acid, phenylsalicylic acid esters and 5-amino-1,3-bis-(2-ethylhexyl)-hexahydro-5-methylpyrimidine(hexetidin), even nanoparticulate silver, copper, zinc and copper salts act antimicrobial, whereby synergistic effects occur, in particular in combination with hexetidine and triclosan. Even quarternary ammonium compounds such as, for example, cetylpyridinine chloride, benzalkonium chloride, domiphen bromide and dequalinium chloride can be used. Octapinol, octenidine, sanguinarine, thymol, menthol, clove oil, cinnamon oil, lemongrass oil and chitosan have also shown to be antimicrobially effective.

Substances that are sensitive to oxidation can hereby be used by means of molecular inclusion in combination with glycodextrines.

The weight proportion of ingredient i) relative to the total mass of the retraction material is customarily between 0 to 10%, preferably 0.01 to 5% and especially preferred 0.1 to 3%.

Buffer substances can be used as ingredient j). Examples are sheet silicates which, based on their layer structure and their ability to exchange cations are in a position to accept protons and in this way achieve a buffer effect. Examples are calcium montmorillonites that represent an aluminum sheet silicate of the smectic group, like the product K40 made by SüdChemie AG or types of Bentone made by Elementis. Additional suitable buffer substances are sodium hydrogen carbonate, sodium carbonate, potassium hydrogen carbonate and/or potassium carbonate.

The weight proportion of ingredient j) relative to the total mass of the retraction material is customarily between 0 to 10%, preferably 0.01 to 5%, and particularly preferred 0.1 to 3%.

Further, the retraction material in accordance with the invention can contain additional additives k) such as smell, taste, coloring agents, tensides, emulgators, indicators for sensory adaptation or for visualization or with binding and surface-moistening properties.

Examples of tensides are nonionic tensides, anionic tensides, cationic tensides, amphoteric tensides preferably biocompatible, biologically degradable tensides such as, for example, fatty alcolyl ethoxylates, silicon tensides, glycosidic tensides in weight proportions of 0 to 5% by weight, preferably 0.01 to 4% by weight, especially preferred 0.1 to 3% by weight.

Examples for coloring agents that can be used are soluble coloring agents, pigments, lacquered coloring agents in proportions of 0 to 5% by weight, preferably 0.001 to 3% by weight and particularly preferred 0.01 to 1% by weight.

The retraction materials in accordance with the invention can be formulated as single component as well as multi-component material, preferably as two-component material consisting of components A and B. Two or multi-component materials are used in particular then, when two components of the formulation are not intended to react with each other during storage. Thus, for certain material combinations there is, for example, a risk that the superabsorber particles b) swell prematurely because of certain ingredients and/or they crosslink. This is, for example, the case in combinations with water or with certain alcohols such as glycerin. In such cases, component A is formulated with the superabsorber particles b) for storage, while component B is formulated with the ingredients that influence the superabsorbent particles such as water or glycerin.

According to a preferred embodiment of the present invention, the retraction material is formulated as two-component retraction material, whereby

base component A contains
a) at least superabsorber particles,

and component B
b) at least water and/or alcohols such as glycerin

and whereby c), at least one astringent is contained in base component A and/or component B).

In a further particularly preferred embodiment, the retraction material in accordance with the invention contains at least one of the ingredients a), b), c) and d).

The pasty retraction material in accordance with the invention is characterized by a high swelling and water absorption behavior. This can be characterized by the volume expansion of a strand of the retraction material after adding drops of water. Preferred retraction materials in the form of a strand with a volume of 1 cm³ display, upon adding drops of water, a volume increase of at least 1.6 cm³ per gram of water added, preferably of 1.6 to 20 cm³ per gram of water added and particularly preferred of 8 to 20 cm³ per gram of water added.

Especially preferred are retraction materials that exhibit a volume expansion of more than 30%, preferably of at least 50%, especially of at least 100% upon adding water. This volume expansion is determined as described in Examples 12 to 19.

The pasty retraction material in accordance with the invention is further characterized by a good consistency.

A better understanding of the invention and its additional goals, characteristics and advantages is given by the enclosed schematic drawings, which are only for explanatory purposes. Shown are:

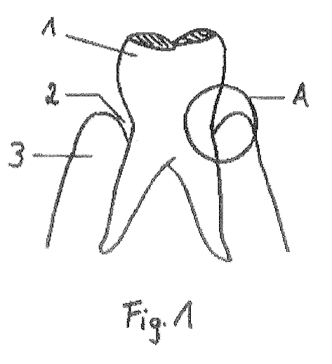

FIG. 1: a schematic view of a cross section of a tooth;

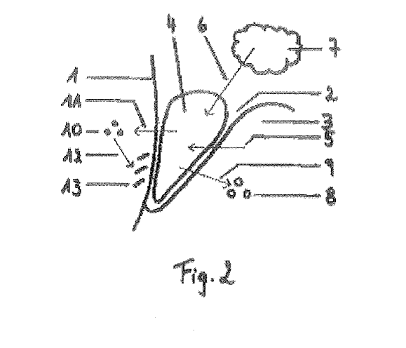

FIG. 2: a detailed cross section of region A of FIG. 1 after inserting the retraction material according to the invention; and

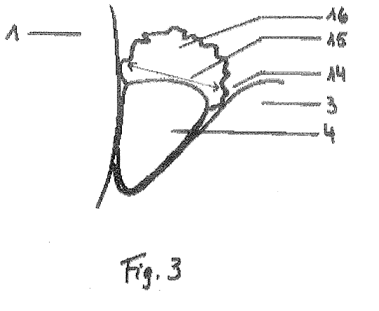

FIG. 3: a detailed cross section of section A of FIG. 1 after widening the gingival sulcus with the retraction material according to the invention.

FIG. 1 shows the gingival sulcus 2, i.e. the pocket-shaped recess between tooth 1 and gingival 3.

FIG. 2 shows the application of a retraction material 4 in a gingival sulcus 2 as detail cross section of region A of FIG. 1. For widening the sulcus 2, the retraction material 4 is inserted into the sulcus 2.

By adding additional ingredients d) to i), the retraction material 4 in accordance with the invention can receive additional desired and advantageous properties.

The retraction material 4 in accordance with the invention contains paste-forming agent a), superabsorber particles b) and astringents c) 8. Superabsorbers are characterized in such a way, that they absorb a multiple of their own weight by absorption 5 and 6 of fluids such as blood from the gingival 3 or saliva and/or water from the mouth upon contact, and thereby swell.

The astringent additives c) 8 work vasoconstricting and hemostatic on the affected gingival tissue 3, by diffusion 9 out of the retraction material 4.

In a preferred embodiment, the retraction material in accordance with the invention contains desensitizers f) 10. Because of the diffusion 11 out of the retraction material 4 into tooth 1, certain selected desensitizers f) 10 can influence the nerve impulse in tooth 1 by mineralization of the hard dental substance 10, e.g. with fluorides and/or by closing 12 the open dentinal tubules 13, e.g. with nanoparticles and thus achieve a desensitization.

FIG. 3 shows the widened gingival sulcus 14 that was created by using the retraction material 4 according to the invention. The superabsorbers contained in the present invention of retraction material 4 offer several advantages. For one, they can, because of their composition, bring about an excellent swelling effect. In addition, they make an excellent compression pressure possible by means of which a high retraction 15 of the sulcus 2 and also, a hemostatic effect is made possible. For another, they are in a position of binding the fluids surrounding the retraction material 4 by jelling 16, so that the retraction material 4 does not flow out.

The following examples are intended to explain the invention without limiting it. Thereby, the indicated percentages respectively relate to the weight proportions, unless otherwise indicated.

### EXAMPLE 1

### Production of a Retraction Paste with polyethylene glycol dimethyl ether as Paste-Forming Agent

The following components were combined in a vacuum mixer and stirred into a homogenous paste:

35% by weight polyethylene glycol dimethyl ether (mol mass 500 g/mol) as paste-forming agent

31% by weight micronized polyacrylic acid sodium salt with an average particle size of 63 μm as superabsorber

24% by weight silicon dioxide with an average particle size of 7 μm as filler

10% by weight aluminum chloride as astringent additive

A viscous retraction material was obtained that can be delivered as a strand of paste by means of a syringe with cannula. The consistency of the material is 29 mm (measured according to ISO 4823 subject to a 15 second load of 1,500 g).

To analyze the swelling and water absorption behavior, a strand of the material (50 mm long, 2.2 mm diameter) that is delivered by the cannula of a syringe, is mixed stepwise with drops of water. Initially, the drops of water are completely absorbed by the retraction material, as a result of which it swells. The initial volume increase of a strand is 1.8 cm³ per gram of water added. After this volume expansion, the material continues to have a compact consistency. Upon further addition of water, starting with two times the weight of the strand of retraction material, water that is dripped onto the strand is no longer absorbed completely, but thickens in the area surrounding the strand. This thickening causes an additional apparent increase of the volume of the strand to a multiple of the original volume of the strand, whereby the material has a sticky, soft consistency at this stage of adding water.

After absorbing water, the retraction material can easily be rinsed off surfaces with a jet of water or with an air/water blower.

This example shows that it is possible in accordance with the invention to provide a retraction material that is easy to apply and can easily be removed after application, which, upon contact with water or watery systems exhibits a significant swelling and thickens surrounding watery fluids. Thus, directly after being applied for widening the sulcus, the material is in a position of binding surrounding blood and saliva.

### EXAMPLE 2

### Production of a Retraction Paste with glycerin as Paste-Forming Agent

The following components were combined in a vacuum mixer and stirred into a homogenous paste:

35% by weight glycerin as paste-forming agent

31% by weight micronized polyacrylic acid sodium salt with an average particle size of 63 μm as superabsorber

24% by weight silicon dioxide with an average particle size of 7 μm as filler

10% by weight aluminum chloride as astringent additive

A viscous retraction material is obtained, which can be delivered as a strand of paste using a syringe with cannula. The consistency of the material is 14 mm (measured according to ISO 4823 subject to a 15 second load of 1,500 g).

To analyze the swelling and water absorption behavior, a strand of the material (50 mm long, 2.2 mm diameter) that is delivered by the cannula of a syringe, is mixed stepwise with drops of water. Initially, the drops of water are completely absorbed by the retraction material, as a result of which it swells. The initial volume increase of a strand is 2.7 cm³ per gram of water added. After this volume expansion, the material continues to have a compact consistency. Upon further addition of water, starting with two times the weight of the strand of retraction material, water that is dripped onto the strand is no longer absorbed completely, but thickens in the area surrounding the strand. This thickening causes an additional apparent increase of the volume of the strand to a multiple of the original volume of the strand, whereby the material has a sticky, soft consistency at this stage of adding water.

After absorbing water, the retraction material can easily be rinsed off surfaces with a jet of water or with an air/water blower.

This example shows that the use of polyols, in particular glycerin, due to the interactions of the hydroxyl groups and the structure of the three-dimensional network, non-continuous, not very sticky strands of thread can be delivered, whereby the material is in a position of widening the sulcus because of its significant swellability, and able to thicken and bind the surrounding blood and saliva.

### EXAMPLE 3

### Production of a Retraction Paste with a Mixture of polyethylene glycol dimethyl ether and glycerin as Paste-Forming Agents

The following components were combined in a vacuum mixer and stirred into a homogenous paste:

25% by weight polyethylene glycol dimethyl ether (mol mass 500 g/mol) as paste-forming agent

10% by weight glycerin as paste-forming agent

31% by weight micronized polyacrylic acid sodium salt with an average particle size of 63 μm as superabsorber

24% by weight silicon dioxide with an average particle size of 7 μm as filler

10% by weight aluminum chloride as astringent additive

A non-sticky, compact retraction material is obtained, which can be delivered as a thread-like strand using a syringe with cannula. The consistency of the material is 22 mm (measured according to ISO 4823 subject to a 15 second load of 1,500 g).

The initial volume increase of the strand (50 mm long, 2.2 mm diameter) is 2.0 cm³ per gram of water added. After this volume expansion, the material continues to exhibit a compact consistency. Upon further addition of water starting with two times of the weight of the strand of retraction material, water that is dripped onto the strand is no longer absorbed completely, but is thickened in the area surrounding the strand. This thickening causes a further apparent expansion of the volume of the strand to a multiple of the original volume of the strand, whereby the material exhibits a sticky, soft consistency at this stage of adding water.

After absorbing water, the retraction material can easily be rinsed off surfaces with a jet of water or with an air/water blower.

This example shows that due to a combination of polyoles, in particular glycerin with polyethers as paste-forming agents, retraction materials can be produced that allow the delivery of non-continuous, not very sticky and easily applicable strands of threads as the result of the interactions of the hydroxyl groups and the building of a three-dimensional network, whereby the material is in a position of widening the sulcus and of thickening and binding surrounding blood and saliva.

### EXAMPLE 4

### Production of a Retraction Paste with a White Oil as Paste-Forming Agent

The following components are combined in a vacuum mixer and stirred into a homogeneous paste:

35% by weight of a hydrocarbon white oil (viscosity at 20° C.: 200 mPas) as paste-forming agent

31% by weight micronized polyacrylic acid sodium salt with an average particle size of 63 μm as superabsorber

24% by weight silicon dioxide with an average particle size of 7 μm as filler

10% by weight aluminum chloride as astringent additive

A viscous retraction material is obtained that can be delivered as a strand of paste by means of a syringe with canulla. The consistency of the material is 28.5 mm (measured according to ISO 4823 subject to a 15 second load of 1,500 g).

To analyze the swelling and water absorption behavior, a strand of the material (50 mm long, 2.2 mm diameter) delivered by a syringe with cannula is subjected stepwise to drops of water. The initial increase in the volume of the strand is 0.5 cm³ per gram of water added. The water that is dripped on is not completely absorbed, but thickened in the area surrounding the strand. This thickening causes a further apparent expansion of the volume of the strand to a multiple of the original volume of the strand, whereby the material exhibits a sticky, soft consistency at this stage of adding water.

After absorbing water, the retraction material can easily be rinsed off surfaces with a jet of water or with an air/water blower.

This example shows that it is possible in accordance with the invention to provide a retraction material that can be applied easily and after application, can be removed again easily, which has a significant viscosity-increasing effect with respect to water that is applied to it or watery systems. Thus, the material is in a position of binding surrounding blood and saliva directly after application into the sulcus.

### EXAMPLE 5

### Production of a Retraction Paste with Silicone Oil as Paste-Forming Agent

The following components were combined in a vacuum mixer and stirred into a homogeneous paste:

35% by weight trimethylsilyl-terminated polydimethylsiloxane with a viscosity at 20° C. of 200 mPas as paste-forming agent

38% by weight micronized polyacrylic acid sodium salt with an average particle size of 63 μm as superabsorber

19% by weight silicon dioxide with an average particle size of 7 μm as filler

8% by weight aluminum chloride as astringent/hemostatic agent

A viscous retraction material is obtained that can be delivered as a strand of paste by a syringe with cannula. The consistency of the material is 28.5 mm (measured according to ISO 4823 subject to a 15 second load of 1,500 g).

To analyze the swelling and water absorption behavior of the material, a strand (50 mm long, 2.2 mm diameter) that is delivered by a syringe with a cannula is subjected stepwise to drops of water. The initial increase in volume of the strand is 0.3 cm³ per gram of water added. The water that is dripped on is not completely absorbed, but is thickened in the area surrounding the strand. This thickening causes a further apparent increase of the volume of the strand to a multiple of the original volume of the strand, whereby the material has a sticky, soft consistency at this stage of adding water.

After absorbing water, the retraction material can be rinsed off surfaces easily with a jet of water or with an air/water blower.

This example shows that it is possible in accordance with the invention to provide a retraction material that is easy to apply and after application, can be removed again easily, which has a significant viscosity-increasing effect on the water that is applied or on watery systems. Thus, directly after application into the sulcus, the material is in a position of binding surrounding blood and saliva.

### EXAMPLE 6

### Production of a Retraction Paste with the Addition of ethanol as Solvent

The following components were combined in a vacuum mixer and stirred into a homogeneous paste:

25% by weight polyethylene glycol dimethyl ether (mol mass 500 g/mol) as paste-forming agent

35% by weight micronized polyactylic acid sodium salt with an average particle size of 63 μm as superabsorber

20% by weight silicon dioxide with an average particle size of 7 μm as filler

10% by weight aluminum chloride as astringent additive

10% by weight ethanol as solvent

A viscous retraction material is obtained which can be delivered as a strand of paste by means of a syringe with cannula. The consistency of the material is 32 mm (measured according to ISO 4823 subject to a 15 second load of 1,500 g).

To analyze the swelling and water absorption behavior, water is dripped stepwise on a strand (50 mm long, 2.2 mm diameter) of the material that is delivered by a syringe with cannula. Initially, the drops of water are completely absorbed by the retraction material, whereby it swells. The initial increase in the volume of the strand is 1.8 cm³ per gram of water added. After this volume expansion, the material continues to have a compact consistency. Upon adding additional water, starting with twice the weight of the strand of the retraction material, water dripped onto the strand is no longer completely absorbed, but thickens in the area surrounding the strand, This thickening causes a further apparent expansion of the strand volume to a multiple of the original volume of the strand, whereby the material has a sticky, soft consistency at this stage of adding water.

After absorbing water, the retraction material can easily be rinsed off surfaces with a jet of water or with an air/water blower.

In addition to the previously mentioned examples, this example shows that the use of solvents leads to a cooling, analgesic and thereby also hemostatic effect, whereby the material is in a position of widening the sulcus and of thickening and binding surrounding blood and saliva.

After absorbing water, the retraction material can easily be rinsed off surfaces with a jet of water or with an air/water blower.

### EXAMPLE 7

### Production of a Retraction Paste with Nanoparticulate hydroxyl apatite as Desensitizing Substance

The following components are combined in a vacuum mixer and stirred into a homogenous paste:

35% by weight polyethylene glycol dimethyl ether (mol mass 500 g/mol) as paste-forming agent

31% by weight micronized polyacrylic acid sodium salt with an average particle size of 63 μm as superabsorber

14% by weight silicon dioxide with an average particle size of 7 μm as filler

10% by weight hydroxyl apatite nanoparticles as desensitizing additive

10% by weight aluminum chloride as astringent additive

A viscous retraction material is obtained that can be delivered as a strand of paste by means of a syringe with cannula. The consistency of the material is 28 mm (measured according to ISO 4823 subject to a 15 second load of 1,500 g).

To analyze the swelling and water absorption behavior, water is dripped stepwise onto a strand of the material (50 mm long, 2.2 mm diameter) that is delivered out of a syringe by means of a cannula. Initially, the drops of water are completely absorbed by the retraction material, whereby it swells. The initial increase in the volume of the strand is 1.8 cm³ per gram of water added. After this volume expansion, the material continues to exhibit a compact consistency. Upon further addition of water starting with twice the weight of the strand of retraction material, water that is dripped onto the strand is no longer completely absorbed, but thickened in the area surrounding the strand. This thickening causes a further apparent increase of the volume of the strand to a multiple of the original volume of the strand, whereby the material has a sticky, soft consistency at this stage of adding water.

After absorbing water, the retraction material can easily be rinsed off surfaces with a jet of water or with an air/water blower.

This example shows that it is possible in accordance with the invention to provide a retraction material that is easy to apply and which can easily be removed after application, which swells significantly upon contact with water or watery systems and thickens surrounding watery fluids. Thus, the material is in a position to widen the sulcus immediately after application and to bind surrounding blood and saliva.

### EXAMPLE 8

### Production of a Retraction Paste with zeolite as Fluid Absorbing Agent

The following components were combined in a vacuum mixer and stirred into a homogeneous paste:

35% by weight polyethylene glycol dimethyl ether (mol mass 500 g/mol) as paste-forming agent

31% by weight micronized polyacrylic acid sodium salt with an average particle size of 63 μm as superabsorber

15% by weight silicon dioxide with an average particle size of 7 μm as filler

9% by weight zeolite in the form of a potassium aluminum silicate with a pore size of 4 angstrom

10% by weight aluminum chloride as astringent additive

A viscous retraction material is obtained that can be delivered as a strand of paste by means of a syringe with a cannula. The consistency of the material is 28 mm (measured according to ISO 4823 subject to a 15 second load of 1,500 g).

To analyze the swelling and water absorption behavior, water is dripped stepwise onto a strand of the material (50 mm long, 2.2 mm diameter) that is delivered out of a syringe by means of a cannula. Initially, the drops of water are completely absorbed by the retraction material, whereby it swells. The initial increase in the volume of the strand is 1.8 cm³ per gram of water added. After this volume expansion, the material continues to exhibit a compact consistency. Upon further addition of water starting with twice the weight of the strand of retraction material, water that is dripped onto the strand is no longer completely absorbed, but thickens in the area surrounding the strand. This thickening causes a further apparent increase of the volume of the strand to a multiple of the original volume of the strand, whereby the material has a sticky, soft consistency at this stage of adding water.

After absorbing water, the retraction material can easily be rinsed off surfaces with a jet of water or with an air/water blower.

This example shows that it is possible in accordance with the invention to provide a retraction material that is easy to apply and which can easily be removed after application, which swells significantly upon contact with water or watery systems and thickens surrounding watery fluids. Thus, the material is in a position to widen the sulcus immediately after application and to bind surrounding blood and saliva.

### EXAMPLE 9

### Production of a Retraction Paste with potassium aluminum sulfate as Astringent and Desensitizing Substance

The following components were combined in a vacuum mixer and stirred into a homogeneous paste:

35% by weight polyethylene glycol dimethyl ether (mol mass 500 g/mol) as paste-forming agent

31% by weight micronized polyacrylic acid sodium salt with an average particle size of 63 μm as superabsorber

24% by weight silicon dioxide with an average particle size of 7 μm as filler

10% by weight potassium aluminum sulfate dodecahydrate as astringent and desensitizing additive.

A viscous retraction material is obtained that can be delivered as a strand of paste by means of a syringe with canulla. The consistency of the material is 29 mm (measured according to ISO 4823 subject to a 15 second load of 1,500 g).

To analyze the swelling and water absorption behavior, water is dripped stepwise onto a strand of the material (50 mm long, 2.2 nun diameter) that is delivered out of a syringe by means of a cannula. Initially, the drops of water are completely absorbed by the retraction material, whereby it swells. The initial increase in the volume of the strand is 1.8 cm³ per gram of water added. After this volume expansion, the material continues to exhibit a compact consistency. Upon further addition of water, starting with twice the weight of the strand of retraction material, water that is dripped onto the strand is no longer completely absorbed, but thickens in the area surrounding the strand. This thickening causes a further apparent increase of the volume of the strand to a multiple of the original volume of the strand, whereby the material has a sticky, soft consistency at this stage of adding water.

After absorbing water, the retraction material can easily be rinsed off surfaces with a jet of water or with an air/water blower.

This example shows that it is possible in accordance with the invention to provide a retraction material that is easy to apply and which can easily be removed after application, which swells significantly upon contact with water or watery systems and thickens surrounding watery fluids. Thus, the material is in a position to widen the sulcus immediately after application and to bind surrounding blood and saliva.

By adding potassium and aluminum ion-containing potassium aluminum sulfate, a hemostatic and a desensitizing effect can be achieved simultaneously.

### EXAMPLE 10

### Production of an Anhydrous Retraction Paste with barium sulfate as X-Ray Opaque Substance

The following components were combined in a vacuum mixer and stirred into a homogeneous paste:

35% by weight polyethylene glycol dimethyl ether (mol mass 500 g/mol) as paste-forming agent

31% by weight micronized polyacrylic acid sodium salt with an average particle size of 63 μm as superabsorber particles

24% by weight nanoparticulate barium sulfate

10% by weight aluminum chloride as astringent additive

A viscous retraction material is obtained that can be delivered as a strand of paste by means of a syringe with cannula. The consistency of the material is 28 mm (measured according to ISO 4823 subject to a 15 second load of 1,500 g).

To analyze the swelling and water absorption behavior, water is dripped stepwise onto a strand of the material (50 mm long, 2.2 mm diameter) that is delivered out of a syringe by means of a cannula. Initially, the drops of water are completely absorbed by the retraction material, whereby it swells. The initial increase in the volume of the strand is 1.8 cm³ per gram of water added. After this volume expansion, the material continues to exhibit a compact consistency. Upon further addition of water starting with twice the weight of the strand of retraction material, water that is dripped onto the strand is no longer completely absorbed, but thickens in the area surrounding the strand. This thickening causes a further apparent increase of the volume of the strand to a multiple of the original volume of the strand, whereby the material has a sticky, soft consistency at this stage of adding water.

After absorbing water, the retraction material can easily be rinsed off surfaces with a jet of water or with an air/water blower.

This example shows, in addition to the previously mentioned examples, that the use of X-ray opaque fillers is possible, which makes residuals of retraction material that remain in the sulcus under unfavorable circumstances visible, by using dental X-ray procedures.

### EXAMPLE 11

### Production of a Very Viscous Retraction Paste by Using Highly Dispersed silica as Paste Thickener and polyethylene glycol dimethyl ether as Paste-Forming Agent

The following components were combined in a vacuum mixer and stirred into a homogeneous paste:

35% by weight polyethylene glycol dimethyl ether (mol mass 500 g/mol) as paste-forming agent

31% by weight micronized polyacrylic acid sodium salt with an average particle size of 63 μm as superabsorber

24% by weight highly dispersed hydrophilic silica with a BET surface of 200 m²/g as paste-thickening agent

10% by weight aluminum chloride as astringent additive

A sticky, compact retraction material is obtained that can be delivered as a thread-like strand by means of a syringe with cannula. The consistency of the material is 13 mm (measured according to ISO 4823 subject to a 15 second load of 1,500 g).

The initial volume increase of the strand (50 mm long, 2.2 mm diameter) is 2.0 cm³ per gram of water added. After this volume expansion, the material continues to have a compact consistency. Upon further addition of water, starting at twice the weight of the strand of retraction material, water that is dripped onto the strand is no longer fully absorbed, but thickens in the area surrounding the strand. This thickening causes a further apparent increase of the volume of the strand to a multiple of the original strand volume, whereby the material has a sticky, soft consistency at this stage of adding water.

After absorbing water, the retraction material can be rinsed off surfaces easily with a jet of water or with an air/water blower.

This example shows that by combining polyether as paste-forming agent with highly dispersed silica as paste thickener, very viscous retraction materials can be produced that allow the delivery of non-continuous, not very sticky and easily applicable strands of threads, whereby the material is in a position of widening the sulcus and of thickening and binding surrounding blood and saliva.

Examples of applications: The application and the sulcus-widening effect of the retraction materials described in examples 1-11 was tested on a gingival model (www.astucesde.com). Thereby, it was shown that all pastes described by way of example were in a position of widening the gingival sulcus and to thus satisfy their suitability as retraction materials.

The retraction materials described in the following examples were characterized by the determination of viscosity, flow limit, consistency and swelling behavior. These measurements were determined as follows:

Viscometry:

The rheologic data of the flow limit and the viscosity of the retraction materials were measured at 20° C. using a Malvern Gemini 200 rheometer. A plate-plate measuring system was used, whereby the upper and the lower plate respectively had a V-shaped contour, profiled crosswise at an angle of 90°, so that pyramidal profile tips were present. The profile had a depth of 0.2 mm and was configured in such a way that approximately 20 recesses or pyramid-like tips were present at a distance of 1 cm. The plates had a diameter of 15 mm. The lower plate was controlled at a temperature of 20° C. by a peltier element at a precision of ±0.1 C.°. During the measurement, the gap distance of the plates was 1 mm. The sample inserted into the gap was shear-mapped in rotation at a shear stress ramp of 0 to 50,000 Pa within 10 minutes. As flow limit, that shear stress was analyzed, at which a maximum occurred in the affiliated viscosity deformation curves. As viscosity measure of the retraction material, the viscosity at a shear stress of 9,000 Pa was used.

Measurement of Consistency:

The consistency was measured according to ISO 4823, with the measurement equipment indicated there and reviewed and analyzed as per the information provided there. The weight was 1,500 g and the load was maintained for 15 seconds. The measurement results were stated in mm of disc diameter. Hereby, viscous materials had smaller measured values, thin-flowing materials, on the other hand, large measured values.

Swelling Behavior:

To measure the swelling behavior of the retraction materials, 0.5 ml of the material is placed in a scalable measuring cylinder. Demineralized water is added little by little, until a permanent layer of excess fluid is achieved. The cylinders are covered with polyethylene film and left overnight at room temperature until equilibrium has been established. The expansion volume of the paste can be read directly off the cylinder scale. The swelling is stated as percent increase of the volume with respect to the initial condition prior to swelling.

Relative Water Absorption:

To measure relative water absorption, demineralized water was added stepwise to 1.0 g of the material in a glass container. Each addition was followed by a 30 minute waiting period in which it was observed whether an excess fluid layer had formed or if the water had been absorbed completely. In the event the water was completely absorbed, additional water was added, In the event that a layer of excess fluid remained, the material was left to stand an additional 60 minutes and excess water was removed with an absorbent cloth.

The relative water absorption was calculated as the relationship of the quantity of water absorbed by the material to the quantity of retraction material that had been weighed in initially.

### EXAMPLE 12

### Production of a Swellable Retraction Paste

The following components were combined in a vacuum mixer and stirred into a homogeneous paste:

27.9% by weight polyethylene glycol dimethyl ether (mol mass 500 g/mol) as paste-forming agent

29.8% by weight micronized polyacrylic acid sodium salt with an average particle size of 63 μm as superabsorber particles

32.7% by weight silicon dioxide with an average particle size of 7 μm as filler

9.6% by weight potassium aluminum sulfate as astringent additive

A viscous retraction material was obtained that could be delivered as a strand of paste out of a capsule by means of a canulla with an interior diameter of 1 mm with the help of a manual applicator. The viscosity of the retraction material at a shear stress of 9,000 Pa at 20° C. was 1.47*10⁷ Pa s. The flow limit was found to be at 2,410 Pa. The consistency measurement resulted in a value of 19 mm. Upon contact with water, the material exhibited a strong increase in volume within 30 seconds up to five times of its initial volume. Parallel to the volume expansion, water that was dripped onto the strand was thickened in the area around the strand. This thickening caused a further apparent increase of the volume of the strand to a multiple of the original volume of the strand, whereby the material had a sticky, soft consistency at this stage of adding water. The swelling of the material with demineralized water was 433%. After absorbing water, the retraction material could easily be rinsed off surfaces with a jet of water or with an air/water blower.

This example shows that it is possible in accordance with the invention to provide a retraction material that is easy to apply, which has a significant volume expansion upon contact with water or watery fluids as the result of the swelling of the superabsorber particles and additionally, thickens surrounding watery fluids. The material is thus in a position of widening the sulcus directly after application and to bind surrounding blood or saliva. As the result of adding astringents, a hemostatic effect is achieved. By using coarse grain filler particles and as the result of the swelled up superabsorber particles, the retraction material can be removed again easily from the sulcus after application, as the large particles are easily picked up and rinsed off by the jet of water or the air/water jet.

### EXAMPLE 13

### Production of a Swellable Retraction Paste

The following components were combined in a vacuum mixer and stirred into a homogeneous paste:

27.68% by weight polyethylene glycol dimethyl ether (mol mass 500 g/mol) as paste-forming agent

27.68% by weight micronized polyacrylic acid sodium salt with an average particle size of 63 μm as superabsorber

30.36% by weight silicon dioxide with an average particle size of 7 μm as filler

5.36% by weight bentonite as filler

8.93% by weight aluminum chloride

A viscous retraction material that could be delivered as a strand of paste out of a capsule with a canulla with an interior diameter of 1 mm with the help of a manual applicator was obtained. The viscosity of the retraction material at a shear stress of 9,000 Pa at 20° C. was 4.70*10⁸ Pa s. The flow limit was found to be at 1,180 Pa. The consistency measurement resulted in a value of 10.5 mm. Upon contact with water, the material showed a strong volume expansion up to five times of its initial volume within 30 seconds. Parallel to the volume expansion, water that is dripped onto the strand is thickened in the area surrounding the strand. This thickening causes a further apparent increase in the volume of the strand to a multiple of the original volume of the strand, whereby the material has a sticky, soft consistency at this stage of adding water. The swelling of the material with demineralized water was 400%. After absorbing water, the retraction material can easily be rinsed off surfaces with a jet of water or with an air/water blower.

This example shows that it is possible in accordance with the invention to provide a retraction material that is easy to apply, which upon contact with water or watery fluids exhibits a significant volume expansion as the result of the swelling of the superabsorber and additionally thickens surrounding watery fluids. Thus, the material is in a position to widen the sulcus immediately after application and to bind surrounding blood and saliva. As the result of the addition of astringents, a hemostatic effect is achieved. By using coarse grain filler particles and because of the swelled up superabsorber particles, the retraction material can easily be removed again from the sulcus, as the coarse particles are easily picked up by the jet of water or air/water jet and are flushed out. The proportion of betonite as sheet silicate ensures that in spite of the high filler content, a paste-like consistency and a plasticity of the paste can be achieved, which is due to a certain sliding effect of the layers of the sheet silicate.

### EXAMPLE 14

### Production of a Swellable Retraction Paste with barium sulfate as X-Ray Opaque Filler

The following components were combined in a vacuum mixer and stirred into a homogenous paste:

26.47% by weight polyethylene glycol dimethyl ether (mol mass 500 g/mol) as paste-forming agent

30.39% by weight micronized polyacrylic acid sodium salt with an average particle size of 63 μm as superabsorber particles

33.33% by weight barium sulfate with an average particle size of 4 μm as filler

9.80% by weight potassium aluminum sulfate as astringent additive.

A viscous retraction material that could be delivered as a strand of paste out of a capsule with a canulla with an interior diameter of 1 mm with the help of a manual applicator was obtained. The viscosity of the retraction material at a shear stress of 9,000 Pa at 20° C. was 7.75*10⁷ Pa s. The flow limit was found to be at 1,630 Pa. The consistency measurement resulted in a value of 12 mm. Upon contact with water, the material exhibits a strong volume expansion, approximately five times that of its initial volume within 30 seconds. Parallel to the volume expansion, water that is dripped onto the strand is thickened in the area surrounding the strand. This thickening causes a further apparent increase of the strand volume to a multiple of the original strand volume, whereby the material has a sticky, soft consistency at this stage of adding water. The swelling of the material with demineralized water was 540%. After absorbing water, the retraction material can easily be rinsed off surfaces with a jet of water or with an air/water blower.

This example shows that it is possible in accordance with the invention to provide a retraction material that is easy to apply, which upon contact with water or watery fluids exhibits a significant volume expansion as the result of the swelling of the superabsorber particles, and additionally thickens surrounding watery fluids. Thus, the material is in a position to widen the sulcus immediately after application and to bind surrounding blood and saliva. As the result of the addition of astringents, a hemostatic effect is achieved. By using coarse grain filler particles and because of the swelled up superabsorber particles, the retraction material can easily be removed again from the sulcus after application, as the coarse particles are easily picked up by the jet of water or air/water jet and are flushed out. Should retraction material inadvertently remain in the sulcus during the rinsing process, it can be recognized on X-rays due to the barium sulfate it contains, and can be removed in targeted manner.

### EXAMPLE 15

### Production of a Swellable Retraction Paste with potassium aluminum sulfate as Filler that can be Dissolved Away

The following components were mixed in a vacuum mixer and stirred into a homogenous paste:

25% by weight polyethylene glycol dimethyl ether (mol mass 500 g/mol) as paste-forming agent

31% by weight micronized polyacrylic acid sodium salt with an average particle size of 63 μm as superabsorber

44% by weight potassium aluminum sulfate as astringent additive and as filler that can be dissolved away.

A viscous retraction material was obtained that could be delivered as a strand of paste out of a capsule by means of a cannula with an interior diameter of 1 mm with the help of a manual applicator. The viscosity of the retraction material at a shear stress of 9,000 Pa at 20° C. was 2.18*10⁸ Pa s. The flow limit was determined to be at 15,000 Pa. The consistency measurement resulted in a value of 10 mm. Upon contact with water, the material exhibits a strong volume expansion of up to three times its initial volume within 30 seconds. Parallel to the volume expansion, water that is dripped onto the strand is thickened in the area surrounding the strand. This thickening causes a further apparent increase of the volume of the strand to a multiple of the original strand volume, whereby the material has a sticky, soft consistency at this stage of adding water. The swelling of the material with demineralized water was 200%. After absorbing water, the retraction material can be rinsed off surfaces very easily with a jet of water or with an air/water blower.

This example shows that it is possible in accordance with the invention to provide a retraction material that is easy to apply, which exhibits a significant volume expansion upon contact with water or watery liquids due to the swelling of the superabsorber particles and additionally, thickens surrounding fluids. Thus, the material is in a position to widen the sulcus immediately after application and to bind surrounding blood and saliva. As the result of the addition of potassium aluminum sulfate as astringent, a hemostatic effect is achieved. By using larger quantities of the crystalline, easily water-soluble potassium aluminum sulfate and due to the swelled superabsorber particles, the retraction material can be removed from the sulcus very easily after application, as the coarse particles are easily picked up by the jet of water or by the air/water jet, dissolved and rinsed off, as in particular because of the dissolving process of the potassium aluminum sulfite, the filler structure of the retraction material breaks down quickly.

### EXAMPLE 16

### Production of a Swellable Retraction Paste

The following components were combined in a vacuum mixer and stirred into a homogeneous paste:

26.74% by weight polyethylene glycol dimethyl ether (mol mass 500 g/mol) as paste-forming agent

26.76% by weight micronized polyacrylic acid sodium salt with an average particle size of 63 μm as superabsorber

29.33% by weight silicon dioxide with an average particle size of 7 μm as filler 5.11% by weight bentonite as filler

8.63% by weight aluminum chloride

3.43% by weight water

A viscous retraction material that could be applied as a strand of paste out of a capsule by means of a canulla with an interior diameter of 1 mm with the help of a manual applicator was obtained. The viscosity of the retraction material at a shear stress of 9,000 Pa at 20° C. was 5.79*10⁷ Pa s. The flow limit was found to be 2,930 Pa. The consistency measurement resulted in a value of 13.5 mm. Upon contact with water, the material exhibits a strong volume expansion of up to five times of its initial volume within 30 seconds. Parallel to the volume expansion, water that is dripped onto the strand is thickened in the area surrounding the strand. This thickening causes a further apparent increase in the volume of the strand to a multiple of the original strand volume, whereby the material has a sticky, soft consistency at this stage of adding water. The swelling of the material with demineralized water was 500%. After absorbing water, the retraction material can easily be rinsed off surfaces with a jet of water or with an air/water blower.

This example shows that it is possible in accordance with the invention, to provide a retraction material that is easy to apply, which exhibits a significant volume expansion upon contact with water or watery liquids as the result of the swelling of the superabsorber particles, and additionally, binds surrounding watery fluids. Thus, the material is in a position to widen the sulcus directly after application and to bind surrounding blood and saliva. By using small quantities of water as paste-forming agent, the viscosity of the retraction paste could be lowered at a simultaneous increase of the flow limit, which further improves the delivery of the material out of narrow canullas. By adding aluminum chloride as astringent, a hemostatic effect is achieved. By using coarse grain filler particles, and as the result of the swelled superabsorber particles, the retraction material can be removed easily from the sulcus after application, as the coarse particles are easily picked up by the jet of water or air/water jet and flushed out.

### EXAMPLE 17

### Production of a Swellable Retraction Paste

The following components were combined in a vacuum mixer and stirred into a homogenous paste:

27.8% by weight polyethylene glycol dimethyl ether (mol mass 500 g/mol) as paste-forming agent

29.7% by weight micronized polyacrylic acid sodium salt with an average particle size of 63 μm as superabsorber

32.6% by weight silicon dioxide with an average particle size of 7 μm as filler

9.6% by weight potassium aluminum sulfate as astringent additive.

0.33% by weight ethereal lemon oil

A viscous retraction material was obtained that could be delivered as a strand of paste out of a capsule by means of a canulla with an interior diameter of 1 mm with the help of a manual applicator. The viscosity of the retraction material at a shear stress of 9,000 Pa at 20° C. was 4.11*10⁶ Pa s. The flow limit was found to be at 1,630 Pa. The consistency measurement resulted in a value of 24 mm Upon contact with water, the material showed a strong increase in volume up to five times of its initial volume within 30 seconds. Parallel to the volume expansion, water that is dripped onto the strand is thickened in the area surrounding the strand. This thickening causes a further apparent increase of the strand volume to a multiple of the original strand volume, whereby the material has a sticky, soft consistency at this stage of adding water. The swelling of the material with demineralized water was 400%. After absorbing water, the retraction material can easily be rinsed off surfaces with a jet of water or with an air/water blower.

This example shows that it is possible in accordance with the invention, to provide a retraction material that is easy to apply, that exhibits a significant expansion upon contact with water or watery fluids as the result of the swelling of the superabsorber particles, and which additionally thickens surrounding fluids. Thus, the material is in a position of widening the sulcus immediately after application and to bind surrounding blood and saliva. Due to the addition of astringents, a hemostatic effect is achieved. By using coarse grain filler particles and as a result of the swelled up superabsorber particles, after application, the retraction material can again be easily removed from the sulcus as the coarse particles are easily picked up by the jet of water or air/water jet and flushed out. The use of ethereal lemon oil increases the plasticity of the retraction material compared to Example 1.

### EXAMPLE 18

### Production of a Swellable Retraction Paste

The following components were combined in a vacuum mixer and stirred into a homogenous paste:

41.7% by weight polyethylene glycol dimethyl ether (mol mass 500 g/mol) as paste-forming agent

8.3% by weight micronized polyacrylic acid sodium salt with an average particle size of 63 μm as superabsorber

41.7% by weight nano-clay (montmorillonite) as filler

8.3% by weight potassium aluminum sulfate as astringent additive.

A viscous retraction material is obtained that could be delivered as a strand of paste out of a capsule by means of a canulla with an interior diameter of 1 mm with the help of a manual applicator. The viscosity of the retraction material at a shear stress of 9,000 Pa at 20° C. was 3.20*10⁸ Pa s. The flow limit was found to be at 6,410 Pa. The consistency measurement resulted in a value of 11.5 mm Upon contact with water, the material showed a strong volume expansion of up to twice its initial volume within 30 seconds. Parallel to the volume expansion, water that is dripped onto the strand is thickened in the area surrounding the strand. This thickening causes a further apparent increase of the strand volume to a multiple of its original strand volume, whereby the material has a sticky, soft consistency at this stage of adding water. The swelling of the material with demineralized water was 200%. After absorbing water, the retraction material can easily be rinsed off surfaces with a jet of water or with an air/water blower.

This example shows that it is possible in accordance with the invention to provide a retraction material that is easy to apply, which exhibits a significant volume expansion upon contact with water or watery liquids as the result of the swelling of the superabsorber particles and additionally, thickens surrounding watery fluids. Thus, the material is in a position to widen the sulcus directly after application and to bind surrounding blood and saliva. By adding astringents, a hemostatic effect is achieved. By using nano-clay and as the result of the swelled up superabsorber particles, the retraction material can easily be removed again from the sulcus after application, as the coarse particles are easily picked up by the jet of water or air/water jet and flushed out.

### EXAMPLE 19

### Production of a Swellable Retraction Paste

The following components were combined in a vacuum mixer and stirred into a homogeneous paste:

41.7% by weight polyethylene glycol dimethyl ether (mol mass 500 g/mol) as paste-forming agent

5.0% by weight micronized polyacrylic acid sodium salt with an average particle size of 63 μm as superabsorber particles

45.0% by weight nano-clay (montmorrilonite) as filler

8.3% by weight potassium aluminum sulfate as astringent additive.

A viscous retraction material was obtained that could be delivered as a strand of paste out of a capsule by means of a canulla with an interior diameter of 1 mm with the help of a manual applicator. The viscosity of the retraction material at a shear stress of 9,000 Pa at 20° C. was 7.99*10⁵ Pa s. The flow limit was found to be at 3,810 Pa. The consistency measurement resulted in a value of 12 mm. Upon contact with water, the retraction material showed a strong volume expansion of up to twice its initial volume within 30 seconds. Parallel to the volume expansion, water that is dripped onto the strand is thickened in the area surrounding the strand. This thickening causes a further apparent increase of the strand volume to a multiple of the original strand volume, whereby the material has a sticky, soft consistency at this stage of adding water. The swelling of the material with demineralized water was 160%. After absorbing water, the retraction material can easily be rinsed off surfaces with a jet of water or with an air/water blower.

This example shows that it is possible in accordance with the invention to provide a retraction material that is easy to apply, which shows a significant volume expansion upon contact with water or watery fluids as the result of the swelling of the superabsorber particles, and additionally thickens surrounding watery fluids. Thus, the material is in a position to widen the sulcus immediately after application and to bind surrounding blood or saliva. By adding astringents, a hemostatic effect is achieved. By using nano-clay, and as the result of the swelled up superabsorber particles, the retraction material can easily be removed from the sulcus after application, as the coarse particles are easily picked up by the jet of water or air/water jet and flushed out.

### COMPARATIVE EXAMPLE 1

### Commercial, Paste-Like Retraction Material “Expasyl™” Manufactured by the Pierre Rolland Company

The very viscous retraction material is delivered as a strand of paste out of a capsule by means of a cannula with an interior diameter of 1 mm with the help of a manual applicator, as instructed by the manufacturer.

The viscosity of the retraction material at a shear stress of 9,000 Pa at 20° C. was 1.41*10⁸ Pa s. The flow limit was found to be at 5,270 Pa. The consistency measurement resulted in a value of 11.5 mm. Drops of water that are dripped onto the strand are not visibly absorbed by the retraction material and the material does not visibly swell within the span of the 10 minutes which are relevant for a retraction treatment, but it partially dissolves at the surface, whereby the dissolved sections have a sticky, soft consistency. Upon further addition of water, the strand is completely macerated. The swelling of the material with demineralized water was 20%. The retraction material can be rinsed off surfaces with a jet of water or with an air/water blower; however, during this process, it sticks relatively strongly to the surfaces.

This example shows that the retraction material Expasyl™ is difficult to apply because of its high viscosity. Because of the high viscosity it is in a position to open a sulcus without flowing away. Upon contact with water or watery systems, however, in contrast to the material in accordance with the invention, no significant volume expansion occurs. Further, the material does not have a thickening effect on surrounding watery fluids. Although it works hemostatically because of its aluminum chloride component, it does, however, not bind saliva. Because of the large quantities of clay minerals it contains, sticky layers of the material remain on the surfaces when being rinsed off.

### COMPARATIVE EXAMPLE 2

### Retraction Material According to Example 1 of DE 690 06 030 T2 with White Clay

The following components were combined in a vacuum mixer and stirred into a homogeneous paste:

66.75% by weight white clay

6.54% by weight aluminum chloride

0.33% by weight ethereal lemon oil

1.02% by weight coloring agent (E 102 and E 131)

The very viscous retraction material is delivered as a strand of paste—as instructed by the manufacturer—out of a capsule by means of a cannula with the help of a manual applicator.

The viscosity of the retraction material at a shear stress of 9,000 Pa at 20° C. was 8.26*10⁸ Pa s. The flow limit was found to be at 2,120 Pa. The consistency measurement yielded a value of 10 mm. Drops of water applied to the strand are not visibly absorbed by the retraction material and the material does not visibly swell within the span of the 10 minutes that are relevant for a retraction treatment, but is partially dissolved at the surface, whereby the dissolved sections have a sticky, soft consistency. Upon further addition of water, the strand is completely macerated. The swelling of the material with demineralized water was 20%. The retraction material can be rinsed off surfaces with a jet of water or with an air/water blower; however, it thereby adheres relatively strongly to the surfaces.

This example shows that the retraction material according to Example 1 of DE 690 06 030 T2 is difficult to apply because of its high viscosity. Because of its high viscosity it is in the position to open the sulcus without flowing away. However, upon contact with water or watery systems, in contrast to the material in accordance with the invention, no significant expansion of volume occurs. Further, the material does not have a thickening effect on surrounding watery fluids. Because it contains aluminum chloride it acts hemostatically, however, it does not bind saliva.

Because it contains large quantities of clay minerals, sticky layers of the materials remain stuck to the surfaces when being rinsed off.

### COMPARATIVE EXAMPLE 3

### Retraction Material According to Example 4 of DE 690 06 030 T2 with Mit Algae Meal

The following components were combined in a vacuum mixer and stirred into a homogenous paste:

46.00% by weight algae meal (alginate powder)

54.00% by weight water

The very viscous retraction material is delivered as a strand of paste—as instructed by the manufacturer—out of a capsule by means of a cannula with the help of a manual applicator.

The viscosity of the retraction material at a shear stress of 9,000 Pa at 20° C. was 8.26*10⁸ Pa s. The flow limit was found to be at 4,060 Pa. The consistency measurement resulted in a value of 19 mm. Drops of water applied to the strand are not visibly absorbed by the retraction material and the material does not visibly swell within the span of the 10 minutes which are relevant for a retraction treatment. Upon further addition of water, the strand is completely macerated. The swelling of the material with demineralized water was 33%. The retraction material can be rinsed off surfaces with a jet of water or with an air/water blower, but it thereby sticks relatively strongly to the surfaces.

This example shows that a retraction material according to Example 5 of DE 690 06 030 T2 is difficult to apply because of its high viscosity. As a result of the high viscosity it is in a position to open a sulcus without flowing away. However, upon contact with water or watery systems, in contrast to material in accordance with the invention, no significant volume expansion occurs. Further, the material does not have a thickening effect upon on the surrounding watery fluids. It is not hemostatic because it does not contain an astringent. The algae meal it contains is not in a position to swell or bind saliva in the short time period of a retraction treatment. Because of the large quantities of algae meal it contains, sticky layers of the material stick to the surfaces while it is being washed off.

TABLE 1
Comparison of Retraction Materials:

| | | | Viscosity | | | | |
|---|---|---|---|---|---|---|---|
| | Consistency | Flow Limit | at 9,000 Pa | Paste Swelling | Relative Water | X-Ray | Ability to |
| | [mm] | [Pa] | [Pa s] | [%] | Absorption [g/g] | Opaqueness | Rinse off |
| Example 12 retraction | 19 | 2,410 | 1.47 * 10⁷ | 433 | 20 | not | + |
| material according to | | | | | | X-ray opaque | |
| the invention | | | | | | | |
| Example 13 retraction | 10.5 | 1,180 | 4.70 * 10⁸ | 400 | 17 | not | + |
| material according to | | | | | | X-ray opaque | |
| the invention | | | | | | | |
| Example 14 retraction | 12 | 1,630 | 7.75 * 10⁷ | 540 | 25 | X-ray opaque | + |
| material according to | | | | | | | |
| the invention | | | | | | | |
| Example 15 retraction | 10 | 15,000 | 2.17 * 10⁸ | 200 | 10 | not | + |
| material according to | | | | | | X-ray opaque | |
| the invention | | | | | | | |
| Example 16 retraction | 13.5 | 2,930 | 5.79 * 10⁷ | 500 | 16 | not | + |
| material according to | | | | | | X-ray opaque | |
| the invention | | | | | | | |
| Example 17 retraction | 24 | 1,630 | 4.11 * 10⁶ | 400 | 18 | not | + |
| material according to | | | | | | X-ray opaque | |
| the invention | | | | | | | |
| Example 18 retraction | 11.5 | 6,410 | 3.20 * 10⁸ | 200 | 6 | not | + |
| material according to | | | | | | X-ray opaque | |
| the invention | | | | | | | |
| Example 19 retraction | 12 | 3,810 | 7.99 * 10⁵ | 160 | 4 | not | + |
| material according to | | | | | | X-ray opaque | |
| the invention | | | | | | | |
| Comparative example 1 | 11.5 | 5,270 | 1.41 * 10⁸ | 20 | 0 | not | ∘ |
| Expasyl ® | | | | | | X-ray opaque | |
| Lot 4988 | | | | | | | |
| Comparative example 2 | 10 | 2,120 | 8.26 * 10⁸ | 20 | 0 | not | ∘ |
| Example 1 of | | | | | | X-ray opaque | |
| DE 690 06 030 T2 | | | | | | | |
| Comparative example 3 | 19 | 4,060 | 2.96 * 10⁷ | 20 | 1 | not | − |
| Example 4 of | | | | | | X-ray opaque | |
| DE 690 06 030 T2 | | | | | | | |

+ can easily be rinsed off with the jet of water
∘ can be rinsed off moderately well with small residuals sticking to surfaces
− difficult to rinse off, strong adhesion

1. Pasty retraction material for widening the gingival sulcus containing
a) a paste-forming agent,
b) superabsorber particles, and
c) at least one astringent additive.

2. Pasty retraction material according to claim 1, characterized by, that ingredient a) is selected from the group consisting of polysiloxanes, polyalkylene oxides, multi-valent aliphatic alcohols, the esters or half-esters of the mentioned Multivalent alcohols with saturated and/or unsaturated carboxylic acids, water or their combinations.

3. Pasty retraction material according to claim 2, characterized by, that the ingredient a) is selected from the group consisting of polysiloxanes, polyethylene glycoles, polypropylene glycoles, polybutylene glycoles, alkanediols, in particular 1,2-propanediol, 1,3-propanediol, 1,2-ethanediol, butanediol and/or hexanediol, of aliphatic triols, in particular glycerin and/or trimethylol propane, or tetrols, pentols or hexols, in particular sorbitol, as well as the alkoxylated derivates of the above mentioned alcohols; of glycerin ester, in particular acetyltributyl citrate, of mineral oils, water or combinations of two or more of these compounds.

4. Pasty retraction material according to claim 1, characterized by, that ingredient b) can absorb at least twice its weight of water at room temperature, preferred is five times its weight, particularly preferred ten times its weight.

5. Pasty retraction material according to claim 1, characterized by, that ingredient b) is selected from the group consisting of micronized and/or nanoparticulate superabsorber particles.

6. Pasty retraction material according to claim 1, characterized by, that ingredient b)
is a polymerizate that is derived from the radical polymerization of one or more ethylenically unsaturated acids and perhaps additional comonomers that can be radically polymerized with it, whereby the share of the ethylenically unsaturated acids in the polymer is at least 55% by weight and/or is a salt of these polymerizates or copolymerizates.

7. Pasty retraction material according to claim 6, characterized by, that ingredient b)
is a polymerizate that is derived from the radical polymerization of one or more ethylenically unsaturated mono or dicarboxylic acids and/or one or more ethylenically unsaturated sulfonic acids, in particular acrylic acid, methacrylic acid, itaconic acid, maleic acid, fumaric acid, vinylsulfonic acid and/or 2-acrylamido-2-methylpropansulfonic acid, and that ingredient b) has an average particle size in the range of 50 nm to 300 μm, preferably 100 nm to 200 μm, particularly preferred of less than 150 μm and very especially preferred, 1 μm to 100 μm
and/or
is a salt of these polymerizates or copolymerizates.

8. Pasty retraction material according to claim 6, characterized by, that ingredient b)
is a crosslinked polymerizate that was obtained by copolymerization in the presence of 0.001 to 5% by weight of crosslinking monomers and in which at least 50% of the acid groups in the polymerizate are carboxylic acid groups and/or
is a salt of these polymerizates or copolymerizates.

9. Pasty retraction material according to claim 1, characterized by, that the superabsorber particles of ingredient b) have an average particle size in the range of 50 nm to 300 μm, preferably 100 nm to 200 μm, particularly preferred of less than 150 μm and very especially preferred 1 μm to 100 μm, and are in particular selected from the group of salts of entirely or partially neutralized polyacrylic acids and/or polymethacrylic acids, especially preferred are their potassium, ammonium and/or sodium salts.

10. Pasty retraction material according to claim 1, characterized by, that such additionally contains organic and/or inorganic filler substance particles d) with a particle size of at least 1 μm, especially filler particles with a particle size in the range of 1 μm and 200 μm, preferably between 1 μm and 100 μm especially preferred between 5 μm and 75 μm and very especially preferred filler particles d) that are selected from the group consisting of aluminum acetate, silicates, sheet silicates, cristobalite, quartz, wollastonite, aluminum oxide, aluminum hydroxide, dental glasses, zinc oxide, titanium oxide, rare earth oxides and fluorides, barium sulfate, zirconium oxide, polymer powder, in particular polyethylene powder, polypropylene powder, polymethyl methacrylate powder, polyurethane powder and/or solid silicone resin, as well as X-ray opaque and fluoride-releasing substances.

11. Pasty retraction material according to claim 10, characterized by, that the filler particles d) are X-ray opaque substances, in particular those substances selected from the group of the oxides of the metals Zn, W, Ag, Yb, Y, Gd, Zr, Sr, Ba, Ta, Nb, Mo, Sn, La, Ce, Pr, Nd, Sm, Eu, Tb, Dy, Ho, Er, Tm, Lu, very especially preferred zirconium dioxide, strontium oxide, bismuth oxide, lanthanum oxide, zinc oxide, yttrium oxide, ytterbium oxide, tungsten oxide, niobium oxide, tantalum oxide, molybdenum oxide, as well as the sulfates of the above mentioned metals, very especially preferred barium sulfate, strontium sulfate, bismuth sulfate, as well as the carbonates of the above mentioned metals, very especially preferred bismuth oxycarbonate and strontium carbonate, as well as the silicates of the metals mentioned above, very especially preferred zirconium silicate, as well as carbides and phosphates of the metals mentioned above, or ytterbium fluoride, bismuth oxychloride, calcium tungstate, barium glasses and strontium glasses, as well as finely distributed metals, in particular zinc, tungsten, silver, ytterbium, yttrium, gadolinium, zirconium, strontium, tantalum, niobium, molybdenum, lanthanum or their alloys.

12. Pasty retraction material according to claim 1, characterized by, that astringents c) are selected from the group consisting of water-soluble aluminum salts, iron salts, zinc salts, alkali halogenides, aluminum ‘acetate, epinephrine, adrenalin, noradrenalin, ephetomin, privin, ortiven, ethacridin, negatol, oxichinolin, tannic acids, tannin, hydrogen peroxide, carbamide peroxide, peroxo salts, calcium salts or the combination of two or more of these compounds and very especially preferred from the group of potassium alum, aluminum chloride, aluminum chlorate, dialuminum pentahydroxy chloride, aluminum hydroxy chloride, aluminum sulfate, aluminum oxalate, aluminum acetate, aluminum lactate, aluminum trifluoromethane sulfonate, aluminum nitrate, aluminum ammonium sulfate, as well as manganese or bismuth salts such as potassium permanganate, bismuth nitrate or bismuth salicylate and other basic, acidic or amphoteric water-soluble aluminum salts, zinc chloride, zinc oxide, zinc salicylate, zinc stearate, zinc sulfate, zinc phosphate, zinc hexafluoro silicate, zinc phenol sulfonate, iron chloride, iron sulfate, sodium chloride, calcium sulfate, calcium sulfate dihydrate, calcium sulfate hemihydrate, calcium hydroxide, calcium hydrogen phosphate, calcium lactate, calcium gluconate and calcium acetate, calcium salts of amino acids, aldonic acids and uronic acids or the combination of two or more of these compounds.

13. Pasty retraction material according to claim 1, characterized by, that such additionally contains fluid-absorbing agents e) that are preferably selected from the group consisting of zeolites, silica gels, alkaline earth and metal sulfates, as well as sheet silicates, very especially preferred from the group consisting of potassium silicate, calcium silicate, sodium silicate, aluminum silicate, zeolites, blue gel, calcium sulfate, aluminum sulfate as well as magnesium sulfate or combinations of two or more of these compounds, or from the group consisting of maltodextrines, alginates, collagens, locust bean gum, chitosanes, agar-agar, carrageenans, guar gum, acacia gum, succino glycane gum, guar meal, tragacanth, karaya gum, xanthan, pectins, cellulose and their ionogenic and non-ionogenic derivatives, in particular carboxymethyl cellulose, hydroxyethyl cellulose or methylhydroxypropyl cellulose, hydrophobically modified cellulose, starch, including modified starch, in particular starch ether, higher molecular polyethylene glycoles and the combinations of two or more of these compounds.

14. Pasty retraction material according to claim 1, characterized by, that such additionally contains desensitizer f) which is preferably selected from the group consisting of potassium salts, strontium salts, eugenol, fluorides, alkali, alkaline earth, and metal phosphates, their polyphosphates, tripolyphosphates, hexametaphosphates, pyrophosphates, hydroxyl phosphates, fluoride phosphates, carbonate phosphates, hydroxyl fluorides phosphates, oxides, silicates, phospho silicates, fluorides, titanates as well as alkali phosphates, tetraalkali pyrophosphates, dialkali hydrogen phosphates and alkali dihydrogen phosphates, very especially preferably the selection is made from the group of potassium chloride, potassium sulfate, potassium carbonate, potassium citrate, potassium acetate, potassium nitrate, strontium chloride, strontium nitrate, strontium citrate, strontium acetate, strontium lactate, eugenol, sodium fluoride, potassium fluoride, stannous fluoride, monofluorophosphates, preferably disodium monofluorophosphate or dipotassium monofluoro phosphate, the fluoride of an organic amino compound, calcium hydroxy phosphate, calcium fluoro phosphate, fluoride-doped hydroxyl apatite represented by the general formula Ca₅(PO₄)₃(OH,Fy) where x and y=1, calcium fluoride, calcium phosphate, preferably amorphous calcium phosphate, calcium sodium phospho silicate, SiG), Zr0₂, ZnO, Ti0₂, aluminum cerium oxide, silica gel functionalized with ammonium chloride, aluminum trifluoride, aluminum(meta)phosphate, aluminum oxide, aluminum silicate, aluminum titanate, tetrasodium and tetrapotassium pyrophosphate, disodium and dipotassium hydrogen phosphate, sodium and potassium dihydrogen phosphate, amorphous calcium phosphate fluoride, amorphous calcium carbonate phosphate, amorphous calcium carbonate phosphate fluoride, amorphous strontium phosphate, amorphous strontium calcium phosphate, amorphous strontium calcium carbonate phosphate and amorphous strontium calcium carbonate phosphate fluoride or combinations of two or more of these compounds.

15. Pasty retraction material according to claim 1, characterized by, that such additionally contains paste thickener g) which is preferably selected from the group consisting of organic paste thickeners, in particular from pentaerythrite, cellulose derivates, polyvinyl pyrrolidon, polyvinyl alcohols, poly(meth)acrylates, xanthan, polyacrylic acid and their salts and esters, mineral oil waxes, polyolefin waxes, polyisobutene waxes, polyether waxes, triglycerides and/or diglycerides, and/or from the group of inorganic paste thickeners, in particular from aluminum hydroxide, zinc oxide, titanium dioxide, zirconium oxide, silicon dioxide, precipitated silica, pyrogenic silica, silanized silica, sheet silicates and combinations of two or more of these compounds.

16. Pasty retraction material according to claim 1, characterized by, that such additionally contains solvent h) which is in particular selected from the group of alcohols, ketones, ether, volatile hydrocarbons, propylene carbonate, as well as water, very especially preferred from the group consisting of ethanol, isopropanol, acetone, ethyl methyl ketone, diethyl ether, ethyl methyl ether, divinyl ether, pentane, hexane, as well as propylene carbonate.

17. Pasty retraction material according to claim 1, characterized by, that such additionally contains disinfecting substances i) preferably selected from the group consisting of phenols, salicyl anilides and salicyl amides as well as their halogenated derivatives, halogenated carbanilides and phydroxybenzoic acid esters, halogenated diphenyl ether, bromochlorophene, bisbiguanides, p-hydroxybenzoic acid, phenyl salicylic acid esters and 5-amino-1,3-bis(2-ethylhexyl)-hexahydro-5-methylpyrimidine, nanoparticulate silver, cooper, zinc and copper salts, quarternary ammonium compounds, octapinol, octenidine and sanguinarine, thymol, menthol, clove oil, cinnamon oil, lemongrass oil, as well as chitosan or combinations of two or more of these compounds, very especially preferably selected from the group consisting of phenol, resorcinols, bisphenols, 2,4-dichloro-2′-hydroxydiphenyl ether, 4,4-dichloro-2′-hydroxydiphenyl ether, 2,4,4′-tribromo-2-hydroxydiphenyl ether, 2,4,4′-trichloro-2′-hydroxydiphenyl ether, chlorhexidine, chlorhexidine gluconate, alexidine, cetylpyridinium chloride, benzalkonium chloride, domiphen bromide and dequalinium chloride.

18. Pasty retraction material according to claim 1, characterized by, that such additionally contains buffer substances j) preferably selected from the group consisting of sheet silicates, which because of their layer structure and their ability to exchange cations are in a position to accept protons and achieve a buffer effect in this way, of sodium hydrogen carbonate, sodium carbonate, potassium hydrogen carbonate and/or potassium carbonate.

19. Pasty retraction material according to claim 1, characterized by, that such additionally contains further additives k), that are selected from the group consisting of aroma, taste, coloring agents, tensides, emulgators, indicators for sensory adaption and/or for visualization and/or with binding and surface-moistening properties or combinations of two or more of these combinations.

20. Pasty retraction material according to claim 1, characterized by, that such contains, relative to the total mixture of retraction material
1 to 90% by weight of ingredient a),
0.1 to 90% by weight of ingredient b),
0.1 to 60% by weight of ingredient c),
0 to 90% by weight of ingredient d),
0 to 80% by weight of ingredient e),
0 to 80% by weight of ingredient f),
0 to 90% by weight of ingredient g),
0 to 40% by weight of ingredient h)
0 to 10% by weight of ingredient i) and/or
0 to 10% by weight of ingredient k).

21. Pasty retraction material according to claim 1, characterized by, that such, in the form of a strand with a volume of 1 cm³, exhibits an increase in volume upon the addition of drops of water of at least 1.6 cm³ per gram of water added.

22. Pasty retraction material according to claim 1, characterized by, that it has a measured viscosity of more than 13,000 Pa s at 20° C. that is determined by using a Malvern Gemini 200 rheometer with a plate-plate measuring system, whereby the upper and the lower plate were respectively profiled with a V-shaped contour cross-wise at 90°, so that pyramidal profile tips with a profile depth of 0.2 mm were present that were attached in such a way that 20 recesses were present in a distance of 1 cm, whereby the plates had a diameter of 15 mm, the gap distance of the plates was 1 mm during measurement and the viscosity was determined at a shear stress of 9,000 Pa.

23. Pasty retraction material according to claim 1, characterized by, that upon the addition of water, such experiences a volume expansion of more than 30%, preferably at least 50%, in particular at least 100%, whereby the volume expansion is determined by inserting 0.5 ml of the material into a scaled measuring cylinder, making multiple additions of demineralized water up to obtaining a permanent excess layer of fluid, letting it stand for twelve hours at room temperature up to the occurrence of equilibrium, reading of the expansion volume and determining the swelling as a percentage increase in volume with respect to the initial condition prior to swelling.

24. Pasty retraction material according to claim 1, characterized by, that such is present in this form in a single or multi-component formulation.

25. Pasty retraction material according to claim 24, characterized by, that the multi-component formulation consists of components A and B, whereby component A contains the superabsorber particles b) and component B the paste-forming agent a), preferably, components of the formulation that represent the swelling and/or crosslinking agents for the superabsorber particles b), in particular water and/or glycerin, and wherein the astringent c) is contained in component A and/or B.

26. Use of a pasty retraction material according to claim 1 for the production of a material for widening the gingival sulcus and for vasoconstriction for the hemostasis of the affected sulcus tissue.
